(19) United States
(12) Patent Application Publication
(10) Pub. No.: US 2013/0344115 A1
(43) Pub. Date: Dec. 26, 2013

(54) METHOD FOR REPAIRING AND PROLIFERATING CARTILAGE TISSUE AND METHOD OF CARTILAGE DEFECT TREATMENT

(71) Applicants: Nai-Jen CHANG, Tainan City (TW); Ming-Long YEH, Tainan City (TW); Chih-Chan LIN, Tainan City (TW)

(72) Inventors: Nai-Jen CHANG, Tainan City (TW); Ming-Long YEH, Tainan City (TW); Chih-Chan LIN, Tainan City (TW)

(21) Appl. No.: 13/607,429

(22) Filed: Sep. 7, 2012

(30) Foreign Application Priority Data
Jun. 22, 2012 (TW) 101122499

(51) Int. Cl. A61K 35/12; A61K 35/14; A61P 19/04; A61K 9/00

(52) U.S. Cl. 424/400; 424/93.7

(57) ABSTRACT

A method for repairing cartilage tissues includes the following steps: adhering a plurality of endothelial progenitor cells onto a scaffold to form a composition, and implanting the composition into an individual. The present invention also provides a method of cartilage defect treatment and a method for proliferating the cartilage tissues. The methods in accordance with the present invention are advantageous for safely and quickly repairing or proliferating cartilage tissues for disease treatment and physical function maintenance.

### CROSS REFERENCE TO RELATED APPLICATIONS

This Non-provisional application claims priority under 35 U.S.C. §119(a) on Patent Application No(s). 101122499 filed in Taiwan, Republic of China on Jun. 22, 2012, the entire contents of which are hereby incorporated by reference.

### BACKGROUND OF THE INVENTION

1. Field of Invention

The present invention relates to a method for repairing or proliferating cartilage tissues and an application of using the same, and more particularly, to a method using a composition containing endothelial progenitor cells and an application using the same.

2. Related Art

Cartilage tissue located at the joint surface of the bones is white and transparent tissue. The cartilage tissue is able to transmit the stress of the bone tissue, absorb the striking force between the bone layer and the joint surface, reduce the friction force of the joint surface and assist the joint in some kind of exercise, like sliding or rolling, with the muscle and ligament tissue. Thus, the cartilage tissue is able to protect the bone tissue in the joint, and further mitigating the wearing of the bone as bearing the foreign stress.

However, the cartilage tissue is a connective tissue without blood vessel, lymphatic system, and nerve. It is mainly composed of hyaline cartilage, type II collagen and proteoglycans. Once the cartilage tissue is damaged, it is hard to be repaired due to the limited amount of the chondrocyte nearby. Even more, the covering of the extracellular matrix makes it hard for the chondrocyte to reach the damaged area.

What currently known is, the repairing reaction will occur as the damage reaching the subchondral bone. However, most of the newly formed tissues are fibrocartilage tissue mainly composed of type I collagen. Because the fibrocartilage tissue lacks the bio-mechanical feature of the cartilage tissue and the function of the hyaline cartilage, it will be gradually degraded. Furthermore, the fibrocartilage tissue is unable to assist the bone recovering to the condition before damage.

The method for repairing the cartilage tissues is different with the level of the damage of the cartilage tissues. Physical therapy, oral medication, steroid are used for patients with minor ailments to ease the pain and swelling of the joint.

For those patients with wearing cartilage tissues, injection of hyaluronic acid and drilling method are used. But for those with severe wearing cartilage tissues, surgery or even arthroplasty is probably a more effective method. However, the lifespan of metal joint is limited. In most of the cases, another arthroplasty or surgery will be needed.

In recent years, the development of tissue engineering, like osteochondral grafting or chondrocyte implantation for cartilage repairing is fast. However, both of the two methods use invasive methods to take the cartilage tissues from other part of the body, and further sending the tissues or cells into the affected area by surgery which cause the other damage to the donating source. That means, patients with damaged cartilage tissues have to experience the pain of surgeries at least twice. Moreover, the defect and degeneration of the donating source or the uneven distribution may occur. In addition, the in-vitro cell culture takes 3 to 4 weeks during the treatment. Patients need to spend a long time waiting and experiencing torture. More importantly, the cells from the above source mostly form fibrocartilage cells mainly composed of type I collagen rather than the hyaline cartilage having type II collagen needed by the joint cartilage. Hence, its repairing effect is limited.

Therefore, it is an important subject to provide a method for repairing the cartilage tissues and achieving the above-mentioned repairing in short time. Compared to the prior art, the method is advantageous for low-invasive and higher forming ratio of hyaline cartilage, and further approves the efficacy and the scope of the cartilage repair and treatment by applying the tissue engineering.

### SUMMARY OF THE INVENTION

In view of the foregoing, it is an object of the present invention to provide a method for repairing the cartilage tissues and achieving the above-mentioned repairing in short time. Compared to the prior art, the method is advantageous for low-invasive and higher forming ratio of hyaline cartilage, and further approves the efficacy and the scope of the cartilage repair and treatment by applying the tissue engineering.

To achieve the above, the present invention discloses a method for repairing the cartilage tissues including the following steps: adhering a plurality of endothelial cells onto a scaffold to form a composition; and implanting the composition into an individual.

In one embodiment of the present invention, the method further includes the step of inducing the cartilage tissues and their peripheral bone tissues of the individual to grow by the endothelial progenitor cells.

In one embodiment of the present invention, before the step of adhering the endothelial cells on the scaffold, the method of the present invention further includes the step of taking the endothelial progenitor cells from blood of the individual for culture. The endothelial progenitor cells are cultured in vitro within one week.

In one embodiment of the present invention, before the step of implanting the composition into the individual, the method of the present invention further includes the step of culturing the endothelial progenitor cells on the scaffold. The endothelial progenitor cells are cultured on the scaffold within one day.

In one embodiment of the present invention, the composition is implanted into the adjacency between the cartilage tissue and its peripheral bone tissue of the individual.

In one embodiment of the present invention, the scaffold is a bio-compatible substance.

In one embodiment of the present invention, the material of the scaffold comprises polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

To achieve the above, the present invention also provides a method of cartilage defect treatment including the following steps: adhering a plurality of endothelial progenitor cells on a scaffold to form a composition, and implanting the composition into an individual.

In one embodiment of the present invention, the method further includes the step of inducing the cartilage tissues and their peripheral bone tissues of the individual to grow by the endothelial progenitor cells.

In one embodiment of the present invention, before the step of adhering the endothelial cells on the scaffold, the method of the present invention further includes the step of taking the endothelial progenitor cells from blood of the individual for cell culture. The endothelial progenitor cells are cultured in vitro within one week before implanting into the scaffold.

In one embodiment of the present invention, before the step of implanting the composition into the individual, the method of the present invention further includes the step of culturing the endothelial progenitor cells on the scaffold. The endothelial progenitor cells are cultured on the scaffold within one day.

In one embodiment of the present invention, the composition is implanted into the adjacency between the cartilage tissue and its peripheral bone tissue of the individual.

In one embodiment of the present invention, the scaffold is a bio-compatible substance.

In one embodiment of the present invention, the material of the scaffold comprises polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

To achieve the above, the present invention also provides a method for proliferating the cartilage tissues including the following steps: adhering a plurality of endothelial progenitor cells on a scaffold to form a composition, and implanting the composition into the adjacency between the cartilage tissues and their peripheral bone tissue.

The word “repair” used here is collectively referred to an action of recovering, maintaining or improving the function of biological tissues by some materials or means. Preferably, repairing means the action to recover, maintain or improve the function of the damaged biological tissues.

As mentioned above, the method for repairing and proliferating cartilage tissue and the method of cartilage defect treatment provided by the present invention are applied by implanting a composition formed by endothelial cells and a cell scaffold to an individual. The composition is for repairing or proliferating the cartilage tissues. Because of the simple way to harvest the endothelial progenitor cells by drawing blood, this method is able to ease the burden of invasive surgery and the pain from drawing marrow in the past. In addition, the composition composed of the endothelial progenitor cells is advantageous for short culturing time and the small amount of cells demand. This is able to shorten the treatment of cartilage repairing.

Furthermore, the method for repairing and proliferating cartilage tissue and the method of cartilage defect treatment have the following advantages. First, the endothelial progenitor cells are taken from the patient himself to prevent from the infection of the immune rejection in the allograft or the xenograft. Second, the composition is able to induce the cartilage tissues and their peripheral bone tissues to grow and repair. Furthermore, forming hyaline cartilage is able to raise the effect of repairing and to recover the whole joint function of patients. Compared to the conventional techniques, the method provided by the present invention are advantageous for decreasing the surgery times, shortening the treatment and repairing the cartilage tissues fast and effectively.

### BRIEF DESCRIPTION OF THE DRAWINGS

The present invention will become more fully understood from the subsequent detailed description and accompanying drawings, which are given by way of illustration only, and thus are not limitative of the present invention and wherein:

FIG. 1 is a flow chart showing the steps of the method for repairing cartilage tissues according to the first embodiment of the present invention;

FIG. 2A is a schematic view of a composition according to one embodiment of the present invention;

FIG. 2B is a photo showing the composition according to one embodiment of the present invention;

FIG. 3 is a flow chart showing the steps of the method for repairing cartilage tissues according to a preferable embodiment of the present invention;

FIG. 4 is a flow chart showing the steps of the treatment method for defect cartilage tissues according to one embodiment of the present invention;

FIG. 5 is a flow chart showing the steps of the method for repairing cartilage tissues according to one embodiment of the present invention;

FIG. 6 is a flow chart showing the steps of the method for proliferating cartilage tissues according to one embodiment of the present invention;

FIG. 7 is an experimental result of the adherence of the endothelial progenitor cells onto the PLGA scaffold;

FIG. 8 is a schematic view showing the appearance of the cartilage tissue repaired by the methods of the present invention

FIG. 9 shows some images of the cartilage tissue repaired by the methods of the present invention observed by micro computed tomography (microCT); and

FIG. 10 is the result of the type II collagen included by the repaired cartilage tissue observed by immuno-histochemistry stain.

### DETAILED DESCRIPTION OF THE INVENTION

The present invention will be apparent from the following detailed description, which proceeds with reference to the accompanying drawings, wherein the same references relate to the same elements.

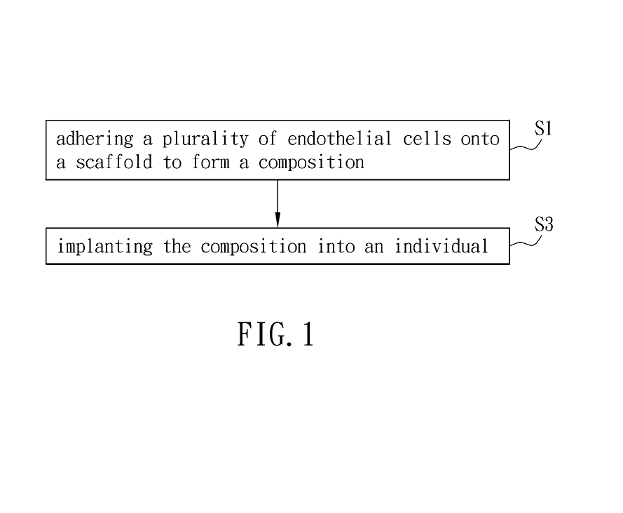

FIG. 1 is a flow chart showing the steps of the method for repairing cartilage tissues. With reference to FIG. 1, in this embodiment, the method for repairing cartilage tissues includes the following steps: adhering the endothelial cells on a scaffold to form a composition (S11), and implanting a composition into an individual. (S13)

To more explicitly illustrate the details of the methods of the present invention, the following provides a clear introduction of the applications, structure, and the manufacturing method of the composition formed by the endothelial progenitor cells and the scaffold. Then, the method of the present invention will be specifically demonstrated based on the composition. However, the following description is for explicitly explanation, and is not meant to be construed in a limiting sense.

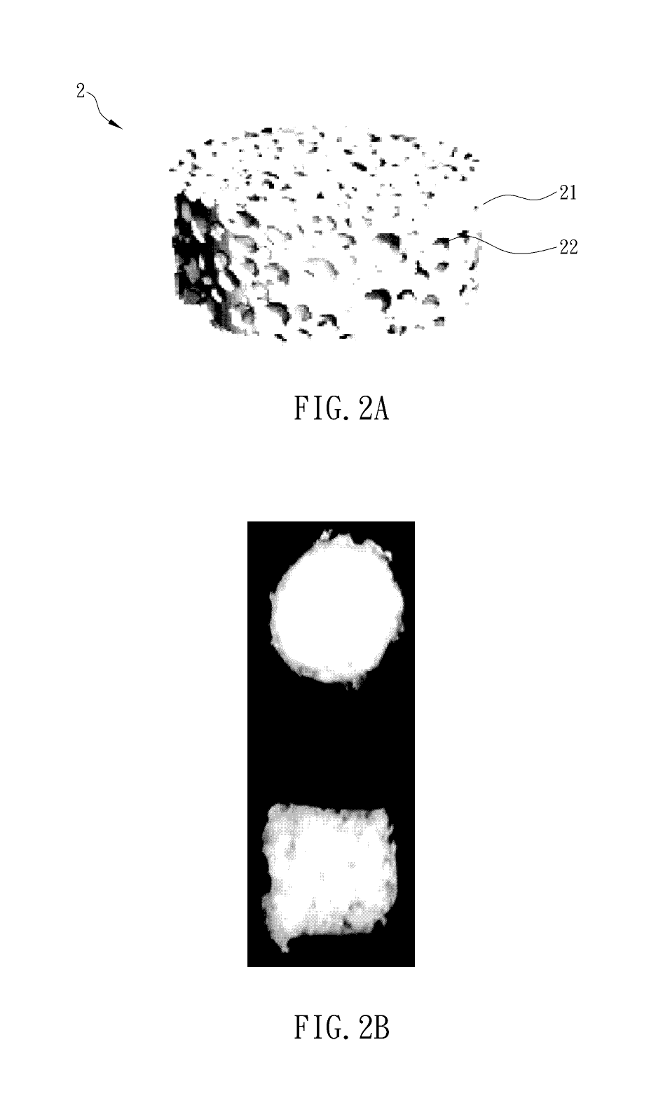

FIG. 2A is a schematic view of a composition according to one embodiment of the present invention. FIG. 2B is a photo showing the composition according to one embodiment of the present invention. The composition drafted in FIG. 2A is for convenient description. The real view of the composition is referred to FIG. 2B. With reference to FIG. 1, FIG. 2A and FIG. 2B, in the step S11, the composition 2 used for repairing cartilage tissues includes a scaffold 21 and a plurality of endothelial progenitor cells 22. The endothelial cells 22 are taken from an appropriate method, like the isolation of mononuclear layer from whole blood, to adhere on the scaffold 21.

The implantation of the composition 2 in step S13 of the present invention is proceed on the premise of the restoration and improvement the condition of the defected cartilage tissues. After the implantation of the composition 2, the newformation of the cartilage tissues may occurs in the damaged area, or the cartilage tissues may fill and repair the damaged area.

The composition 2 is for implantation. The word “implantation” used here is also collectively referred to other method capable of bringing the composition 2 to the position adjacent to the site to be repaired, and the present is not limited to this method. When the composition 2 is implanted into an individual, it is preferably implanted into the position adjacent to the cartilage tissues to be repaired or the adjacency of the cartilage tissues and its peripheral bone tissues.

The material of Scaffold 21 can be bio-degradable, bio-absorbable, bio-compatible substance or the combination of any substance mentioned above. More specifically, it includes polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan or poly(ethylene glycol) (PEG), but the present invention is not limited to the material. In this embodiment, the material of the scaffold 11 is substantially poly (lactic-co-glycolic) acid (PLGA).

Poly (lactic-co-glycolic) acid (PLGA) is polymerized by poly lactic acid and glycolic acid by different proportion. Practically, the range of the mixing proportion of the poly lactic acid and the glycolic acid is from about 1:1 to about 9:1. More practically, the ratio is 50:50, 60:40, 65:35, 70:30, 75:25, 80:20, 85:15 or 90:10. The more the glycolic acid included in copolymer, the slower the degradation of the copolymer. For example, when the ratio of the poly lactic acid and the glycolic acid is 85:15, the composition 2 containing the formed scaffold 21 can offer better repairing effect.

Otherwise, the scaffold 21 can be coated, cladded or modified by other substances, like growth factors or natural substances, beneficial to the growth of the cells, and the present invention is not limited to this construction. The scaffold 21 is preferably multi-porous scaffold which is also advantageous for the adherence and the growth of the cells.

In this embodiment, before the step S11, the harvest of the endothelial progenitor cells and the in-vitro culture can be preceded first. The endothelial progenitor cells 22 are taken from the blood of an individual which has cartilage tissues to be repaired. Preferably, the cells are taken from the blood of the individual, and then centrifugating for several times to get pure cells. The method of in-vitro culture can be done by the general way of the prior techniques or referred by the experiment example mentioned below. The method is well-understood by the person having ordinary skill in the art, and is not repeated here.

By using the materials and the proportion mentioned above, the scaffold 21 is well-prepared. The manufacturing method of the scaffold 21 is well-understood by the person having ordinary skill in the art, and are not repeated here. Subsequently, mix the blood with HBSS with the ratio 1:2, and centrifugate with the Ficoll-Hypaque method. Then, extract the mononuclear layer and centrifugate for several times. Culture the residual cells onto the dish coated with fibronectin to get endothelial progenitor cells 22.

Next, inject the solution containing endothelial progenitor cells 22 into the scaffold 21, and incubate both of the cells and the scaffold of the step S11 of the present invention, and further form the composition 2. In detail, due to the multi-porous structure of the scaffold, the endothelial progenitor cells 22 can randomly adhere onto the surface of the scaffold 21, the porous structure of the scaffold 21 or the combination of the above, and the present invention is not limited to this construction.

As mentioned above, it takes merely a short time to finish the preparation of the composition 2 after the implantation of the endothelial progenitor cells 22. Compared to the long time the prior techniques takes, the present invention is advantageous for instant application and short-time treatment. Because the composition 2 is injected into the adjacency between the cartilage tissues and its peripheral bone tissues, the composition 2 is able to induce the cartilage tissues and its peripheral bone tissues to grow and move rather than culturing the cells in vitro, and further completing the repairing. Thus, the culturing time of the endothelial progenitor cells 22 on the scaffold 21 is short. Stable adherence is the chief demand. In practical application, the day of culturing can be 1 day, 2 days, 1 week, or 2 weeks, preferably within 1 day. Due to the same reason, the material of the scaffold 21 can be used without the surface modification. For certain, in the better embodiment, the surface of the scaffold 21 can be modified to shorten the time for the endothelial progenitor cells 22 adherence, and the present invention is not limited to the construction.

After forming the composition 2 of the present invention by the methods mentioned above, the implantation can be performed according to the position to be repaired. In detail, in this embodiment, the composition 2 is implanted into the adjacency between the cartilage tissues and the bone tissues. Preferably, the composition 2 is implanted into the adjacency between the hyaline cartilage tissues and its subchondral bone, or the so called damaged osteochondral area. In addition, the word “damaged” used herein is collectively referred to the wearing, softening, crashing or diminishing of the cartilage tissues, and is further causing defect or even splicing of the cartilage tissues. Otherwise, the word “implant” used herein is collectively referred to the action of forming an orifice on the surface of the individual and sending the composition 2 into the preset location. This implantation method is helpful for the medical stuff to precisely place the composition 2 into the correct location and further raise the effect of the repairing. However, the present invention is not limited to the method. In other embodiments, injection method advantageous for saving the time and easing the pain of patients is also available for imputing the composition 2 into the individual.

After implanting into the position to be repaired, the endothelial progenitor cells 22 are able to stay at the implanting location without flowing or leaving by adhering on the scaffold 21. Furthermore, the endothelial progenitor cells 22 induce the cartilage tissues and its peripheral bone tissue to grow and repair. That is, the implantation of the endothelial progenitor cells 22 is able to promote the proliferation, growth and differentiation of the cartilage tissues, and thus generating the hyaline cartilage cells and covering and/or filling the damaged area.

In this embodiment, the endothelial progenitor cells 22 are harvested by drawing blood and purring. That is, there is no invasive action into the organism when the cells are harvested. The individual has only to experience the implantation surgery of the composition 2 once, and thus dispensing the invasive methods, like surgery or marrow drawing, to harvest cells and decreasing the pain and the risk patients may experience.

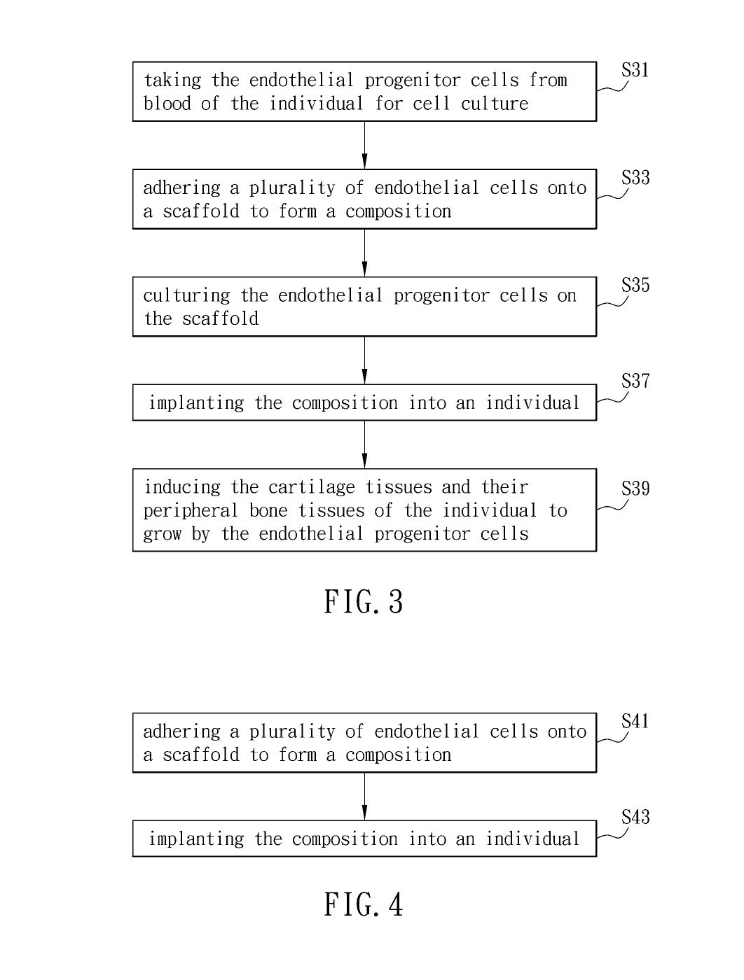

FIG. 3 is a flow chart showing the steps of the method for repairing cartilage tissues according to a preferable embodiment of the present invention. With reference to FIG. 3, in this embodiment, the method for repairing cartilage tissues includes the following steps: taking the endothelial progenitor cells from the blood of an individual (S31), adhering the endothelial cells on a scaffold to form a composition (S33), culturing the endothelial progenitor cells on the scaffold (S35), implanting a composition into an individual (S37) and inducing the cartilage tissues and its peripheral bone tissue by the endothelial progenitor cells (S39). But the techniques and the implementation details of the steps have been disclosed by the above-mentioned description, and are not repeated here.

Particularly, in the step S35 mentioned above, the endothelial progenitor cells are cultured in-vitro for one day, two days, one week or two weeks before implanting into the scaffold. That is, the endothelial progenitor cells harvested from the blood drawn from the organism are able to adhere onto the scaffold with short-time purification and culture. That's because the cells applied by the present invention is used to induce the cartilage tissues and/or their peripheral bone tissues growing, and further completing the repairing. Hence, the amount of the cells used in present invention is small, and the manufacturing time of the composition is effectively shortened.

In addition, same as the description above, in this embodiment, endothelial progenitor cells are able to complete the adherence within one day. Overall, the manufacturing method of the composition can ne completed within seven days. This is advantageous for saving manpower, materials and the treatment time, and accelerating the repairing.

With reference to FIG. 4, the present invention further provides a treatment method for defect cartilage tissues. It includes the following steps: adhering a plurality of endothelial progenitor cells on a scaffold to form a composition (S41), and implanting the composition into an individual (S43).

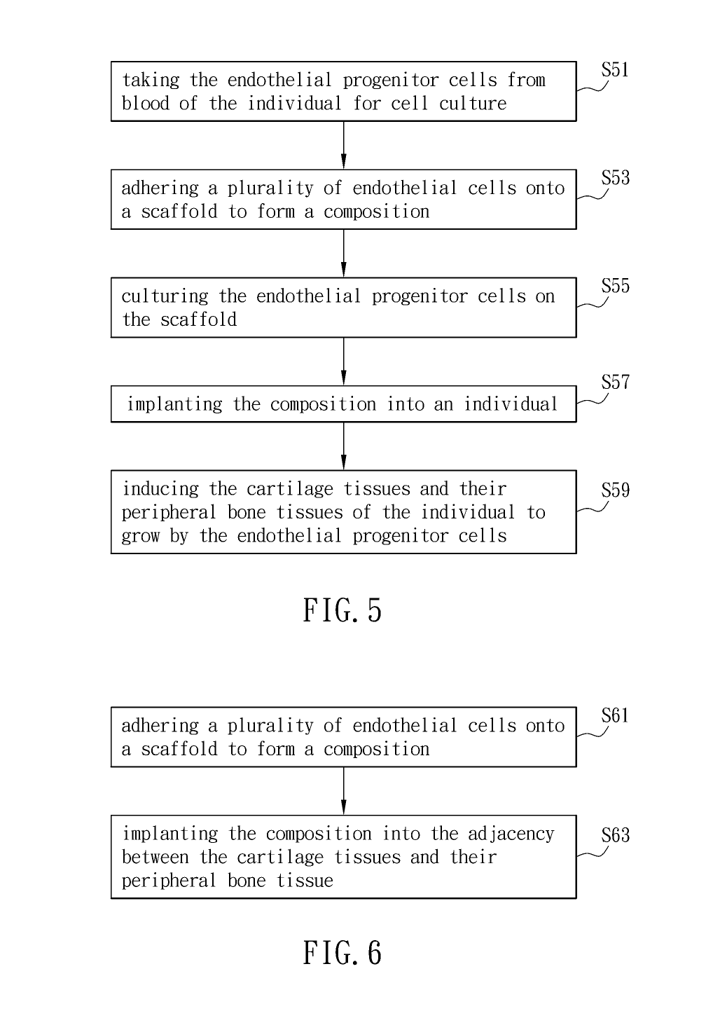

FIG. 5 is a flow chart showing the steps of the method for repairing cartilage tissues according to one embodiment of the present invention. With reference to FIG. 5, in this embodiment, the treatment method of defected cartilage tissues includes: taking the endothelial progenitor cells from the blood of an individual (S51), adhering a plurality of endothelial progenitor cells on a scaffold to form a composition (S53), culturing the endothelial progenitor cells on the scaffold (S55), implanting the composition in to an individual (S57), and inducing the cartilage tissues and its peripheral bone tissue by the endothelial progenitor cells (S59).

The techniques and the implementation details of the steps have been disclosed by the above-mentioned description and referred by the experiment example mentioned below, hence, the details are not repeated here. The word “damaged” used herein is also collectively referred to the wearing, softening, crashing or diminishing of the cartilage tissues, and is further causing defect or even splicing of the cartilage tissues. The word “treatment” used herein is also collectively referred to the action of giving the composition to the individual suffering from or possibly developing into the cartilage tissue defect in order to be radically cured, mitigated, softened, prevented or improved from those symptom, secondary condition, or possibility of damage and disease. The treatment referred by the present invention can be preceded single or with other medicine and cure method, and the present invention is not limited to the method. As for applying the method of the present invention, at least one composition has to be implanted. However, according to the level and the range of the defect practically, one, two, or even more than five compositions might be implanted into the individual, and the present invention is not limited to the amount.

With reference to FIG. 6, the present invention further provides a method for proliferating cartilage tissues. The method includes the following steps: adhering a plurality of endothelial progenitor cells on a scaffold to form a composition (S61) and implanting the composition into the adjacency between the cartilage tissue and its peripheral bone tissue (S63). But the techniques and the implementation details of the steps have been disclosed by the above-mentioned description, and are not repeated here.

The following and accompanying figures take a number of experiments for examples to describe the main details of the method for repairing and proliferating cartilage tissue and method of cartilage defect treatment and the practical applying method and the effect of the implantation of the composition in accordance with the embodiments of the present invention.

Experiment 1: The Seeding of Endothelial Progenitor Cells into the PLGA Scaffold

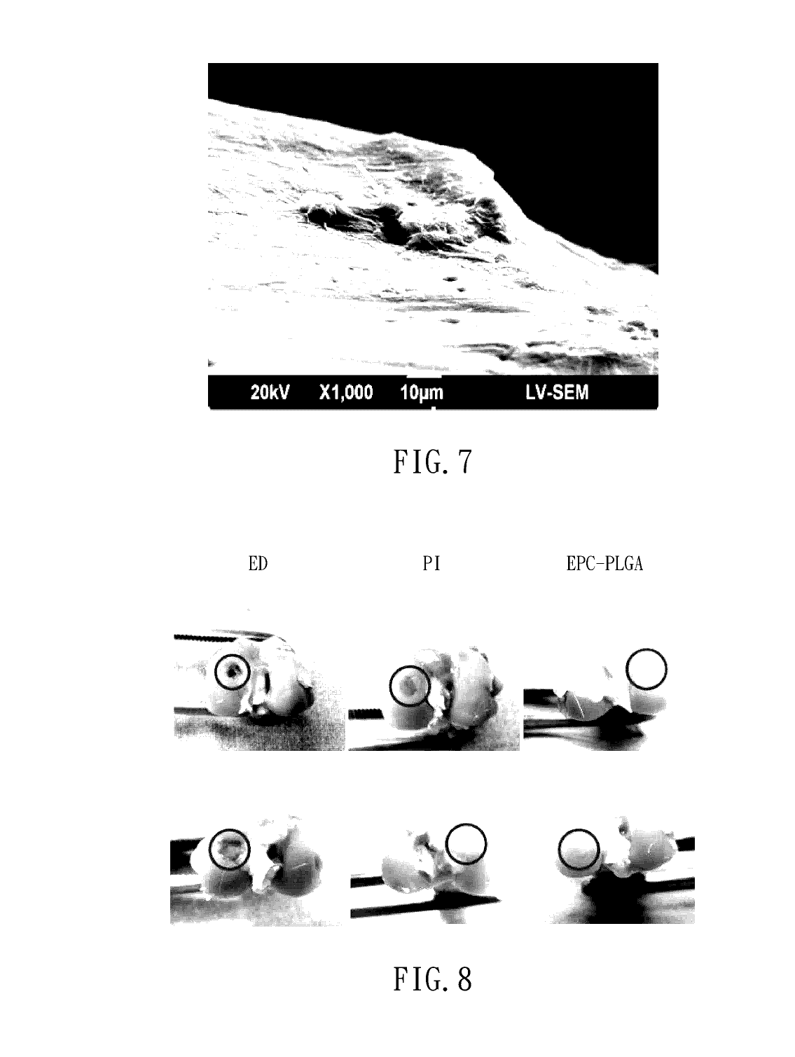

The endothelial progenitor cells were cultured on the dish with 10% trypsin. The PLGA scaffolds were infiltrated in 75% ethanol for 5 minutes. Then, the scaffolds were washed with PBS for 5 times and placed in a 24 well plate and put aside for later usage. The endothelial progenitor cells were cultured in the dish with 10% trypsin. In the meantime, the cells were counted and adjusted the concentration at 5*105 cells/ml. 100 μL of cell solution was injected with syringe. And the solution was checked for fully infiltrating into the scaffold. The dish is incubated in 37° C. for 4 hours. After the 4 hours incubation, 1.5 ml of medium is filled in, and then incubating in 37° C. After 24 hour of cultivation, the scaffold attached with cells is ready for implantation. The result is referred to FIG. 7.

FIG. 7 is an experimental result of the adherence of the endothelial progenitor cells onto the PLGA scaffold. With reference to FIG. 7, where the arrow point is the adhered endothelial progenitor cells on the PLGA scaffolds. This result clearly illustrates that the endothelial progenitor cells and the PLGA scaffolds can be combined together to form the composition of the present invention.

Experiment 2: Surgical Procedures in Osteochondral Model

All surgical procedures were approved by the Animal Care and Use Committee of National Cheng Kung University. Thirty-eight 4-5-month-old New Zealand White male rabbits weighing 2-3 kg were used in this study. Before surgery, anesthesia was induced with a subcutaneous injection of Zoletil 50 (25 mg/kg), followed by intubation and maintenance with a mixture of 2% isoflurane and oxygen/nitrous oxide (1/0.4 L/min) through an automatic ventilator. Under anesthesia, both legs were then shaved, brushed, disinfected with 1% ethanol-iodine, and covered with a drape. The knee was exposed by an anteromedial parapatellar longitudinal and capsular incision. The knee joint was then immobilized in the maximally hyper-flexed position. The patella was dislocated laterally to expose the medial femoral condyle. Therefore, a full-thickness osteochondral defect that was 3 mm in depth and 3 mm in diameter was created with an electric drill on the weight-bearing zone of the medial femoral condyle.

The joint was then irrigated immediately with sterile isotonic saline. After removing the debris from the defect with a curette and cleaning the defect edge with a scalpel blade, the rabbits were allocated randomly into empty defect (ED), PLGA-implanted (PI), and EPC-PLGA groups. A PLGA scaffold that was pre-sterilized in 75% ethanol was inserted gently into the defect hole by press-fit fixation and subsequently flushed with normal saline and repositioned in the patellar position, followed by wound closure. The capsule was closed carefully using 3-0 absorbable Vicryl sutures. The subcutaneous tissues and skin were repaired using 3-0 nylon sutures.

All of the rabbits were housed singly in a stainless-steel cage. An antibiotic (25 mg/kg, Enrofloxacin) and analgesic (Ketoprofene) therapy was administered immediately after each surgery and for 3 days thereafter, and the wounds were dressed with povidone-iodine for 7 days. In addition, in the sham group, no defects were created in the rabbit's knees; the knees were only opened, and the wounds were closed as described above. All of the rabbits were observed for body weight, appetite, wound healing, and proper functional activity after surgery. The rabbits were euthanized 4 or 12 weeks after surgery with an intravenous injection of 120 mg/kg pentobarbital. The result is referred to FIG. 8 to FIG. 10.

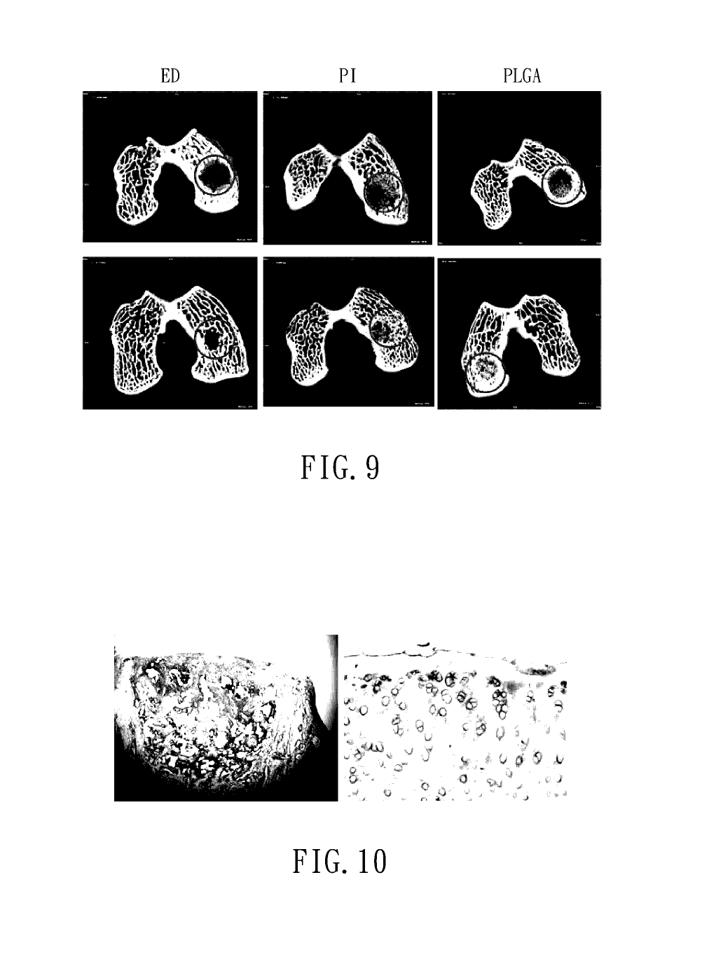

FIG. 8 is the outside view of the cartilage tissue repaired by the methods of the present invention. FIG. 9 is the image of the cartilage tissue repaired by the methods of the present invention observed by micro computed tomography (microCT). With reference to FIG. 8 and FIG. 9, the upper and the bottom row of the images were the fourth week result and the twelfth week result, respectively. From left to right were empty defect (ED) group, PLGA-implanted (PI) group, and EPC-PLGA group. As the image shown, Compared the 3 groups of fourth week condition, the repairing effect of the EPC-PLGA group is much more obvious to the other two groups. For EPC-PLGA group, the damaged area of the cartilage tissues presented better repairing effect. According to the twelfth week result, the effect of the EPC-PLGA group is much more obvious.

FIG. 10 is the result of the type II collagen included by the repaired cartilage tissue observed by immuno-histochemistry stain. With reference to FIG. 10, the left one and the right one individually shows the result of the fourth week and the twelfth week. As the image shown, the type II collagen included by repaired cartilage tissue is as predicted by the present invention. Moreover, the result of twelfth week is much more obvious than the fourth week.

It can be seen that the method of the present invention is able to effectively repair the damaged cartilage tissues, and its effect is much more obvious with the addition of the repairing day.

As mentioned above, the method for repairing and proliferating cartilage tissue and method of cartilage defect treatment provided by the present is applied by implanting a composition formed by endothelial cells and cell scaffold. The method is for repairing or proliferating the cartilage tissues. Because of the simple way to harvest the endothelial progenitor cells by drawing blood, this method is able to ease the burden of invasive surgery and the pain from drawing marrow in the past. In addition, the composition composed of the endothelial progenitor cells is advantageous for short culturing time and the small amount of cell demand. This is able to shorten the treatment of cartilage repairing.

Furthermore, the method for repairing and proliferating cartilage tissue and method of cartilage defect treatment have the following advantages. First, the endothelial progenitor cells are taken from the patient himself to prevent from the infection of the immune rejection in the allograft or the xenograft. Second, the composition is able to induce the cartilage tissues and its peripheral bone tissues to grow and repair; furthermore, forming hyaline cartilage is able to raise the effect of repairing and to recover the whole joint function of patients. Compared to the conventional techniques, the method provided by the present invention are advantageous for decreasing the surgery times, shortening the treatment and achieving the efficacies of fast and effectively repairing the cartilage tissues.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

1. A method for repairing cartilage tissues, comprising the steps of:
adhering a plurality of endothelial progenitor cells onto a scaffold to form a composition; and
implanting the composition into an individual,
wherein the composition is directly pressed into the adjacency between the cartilage tissues and their peripheral bone tissue of the individual, and the endothelial progenitor cells induce the cartilage tissues and their peripheral bone tissue to grow and repair.

2. The method according to claim 1, further comprising the step of:
inducing the cartilage tissues and their peripheral bone tissues of the individual to grow by the endothelial progenitor cells

3. The method according to claim 1, before the step of adhering the endothelial cells on the scaffold, further comprising the step of:
taking the endothelial progenitor cells from blood of the individual for cell culture.

4. The method according to claim 3, wherein the endothelial progenitor cells are cultured in vitro within one week.

5. The method according to claim 1, before the step of implanting the composition into the individual, further comprising the step of:
culturing the endothelial progenitor cells on the scaffold.

6. The method according to claim 5, wherein the endothelial progenitor cells are cultured on the scaffold within one day.

7. (canceled)

8. The method according to claim 1, wherein the scaffold is a bio-compatible substance.

9. The method according to claim 1, wherein the material of the scaffold comprises polycaprolactone (PCL), polylactic acid (PLA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

10. A method of cartilage defect treatment, comprising the steps of:
adhering a plurality of endothelial progenitor cells on a scaffold to form a composition; and
implanting the composition into an individual,
wherein the composition is directly pressed into the adjacency between the cartilage tissues and their peripheral bone tissue of the individual, and the endothelial progenitor cells induce the cartilage tissues and their peripheral bone tissue to grow and repair.

11. The method according to claim 10, further comprising the step of:
inducing the cartilage tissues and their peripheral bone tissues of the individual to grow by the endothelial progenitor cells.

12. The method according to claim 10, before the step of adhering the endothelial cells on the scaffold, further comprising the step of:
taking the endothelial progenitor cells from blood of the individual for cell culture.

13. The method according to claim 12, wherein the endothelial progenitor cells are cultured in vitro within one week.

14. The method according to claim 10, before the step of implanting the composition into the individual, further comprising the step of:
culturing the endothelial progenitor cells on the scaffold.

15. The method according to claim 14, wherein the endothelial progenitor cells are cultured on the scaffold within one day.

16. (canceled)

17. The method according to claim 10, wherein the scaffold is a bio-compatible substance.

18. The method according to claim 10, wherein the material of the scaffold comprises polycaprolactone (PCL), polylactic acid (PIA), polyglycolic acid (PGA), poly(lactic-co-glycolic) acid (PLGA), poly(p-dioxanone), polyanhydride, polyethylene terephthalate (PET), polyorthoester (POE), collagen, gelatin, hyaluronic acid, chitosan, or polyethylene glycol (PEG).

19. A method for proliferating cartilage tissues, comprising the steps of:
adhering a plurality of endothelial progenitor cells on a scaffold to form a composition; and
directly pressing the composition into the adjacency between the cartilage tissues and their peripheral bone tissue,
wherein the endothelial progenitor cells induce the cartilage tissues and their peripheral bone tissue to grow and repair.
